(19) United States
(12) Patent Application Publication
(10) Pub. No.: US 2008/0261864 A1
(43) Pub. Date: Oct. 23, 2008

(54) Combination of nateglinide or repaglinide with at least one further antidiabetic compound

(76) Inventor: Edwin Bernard Villhauer, Morristown, NJ (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 12/157,822

(22) Filed: Jun. 13, 2008

Related U.S. Application Data
(63) Continuation of application No. 10/471,253, filed on Sep. 10, 2003, now abandoned.
(60) Provisional application No. 60/275,098, filed on Mar. 12, 2001.

(51) Int. Cl. A61K 38/28; A61K 31/165; A61K 31/7036; A61K 31/5415; A61P 3/00

(52) U.S. Cl. 514/4; 514/617; 514/42; 514/224.2

(57) ABSTRACT

The present invention relates to a combination of organic compounds which comprises at least two antidiabetic agents, preferably with different modes of action, in which the active ingredients are in each case present in free form or in the form of a pharmaceutically acceptable salt and, optionally, at least on pharmaceutically acceptable carrier, for simultaneous, separate or sequential use.

The generally accepted aims in the treatment of diabetes are to provide relief from symptoms, improvement of the quality of life and prevention of both acute (hyperosmolar coma and ketoacidosis) and chronic complications (e.g. diabetic neuropathy, diabetic nephropathy and premature atherosclerosis). Type 2 diabetes is characterized by both increased peripheral insulin resistance and abnormal insulin secretion. At least two abnormalities of insulin secretion are recognized: in the first phase insulin is both delayed and inadequate in the face of elevated circulating glucose levels and in the second phase insulin secretion is lost. Several metabolic hormonal, and pharmacological entities are known to stimulate insulin secretion including glucose, amino-acids and gastrointestinal peptides. The Diabetes Control and Complications Trial (DCCT) performed in Type I IDDM subjects has established that lowering d blood glucose is associated with decreases in the onset and progression of diabetic microvascular complications (Diabetes Control and Complications Trial Research Group; N. Engl. J. Med. 1993, 329, 977-986). Therefore, one therapeutic focus is on optimizing and potentially normalizing glycemic control in subjects having diabetes. Presently available oral agents needed to be improved in order to better meet this therapeutic challenge.

The present invention relates to a combination, such as a combined preparation or pharmaceutical composition, respectively, which comprises nateglinide of formula (I)

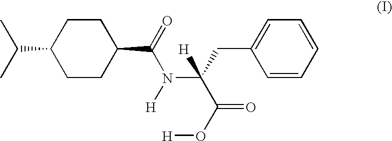

or repaglinide and at least one further antidiabetic compound selected from the group consisting of insulin signalling pathway modulators, like inhibitors of protein tyrosine phosphatases (PTPases), antidiabetic non-small molecule mimetic compounds and inhibitors of glutamine-fructose-6-phosphate amidotransferase (GFAT); compounds influencing a dysregulated hepatic glucose production, like inhibitors of glucose-6-phosphatase (G6Pase), inhibitors of fructose-1,6-bisphosphatase (F-1,6-BPase), inhibitors of glycogen phosphorylase (GP), glucagon receptor antagonists and inhibitors of phosphoenolpyruvate carboxykinase (PEPCK); pyruvate dehydrogenase kinase (PDHK) inhibitors; inhibitors of gastric emptying; insulin; inhibitors of GSK-3; retinoid X receptor (RXR) agonists; agonists of Beta-3 AR; agonists of uncoupling proteins (UCPs); non-glitazone type PPARγ agonists; dual PPARγ/PPARα agonists; antidiabetic vanadium containing compounds; incretin hormones, like glucagon-like peptide-1 (GLP-1) and GLP-1 agonists; β-cell imidazoline receptor antagonists; miglitol; and α₂-adrenergic antagonists; in which the active ingredients are present in each case in free form or in the form of a pharmaceutically acceptable salt and optionally at least one pharmaceutically acceptable carrier; for simultaneous, separate or sequential use, especially in the prevention, delay of progression or treatment of diseases, very especially metabolic disorders and in particular type 2 diabetes mellitus and diseases and conditions associated with diabetes mellitus. Such a combination is preferably a combined preparation or a pharmaceutical composition.

By the term “a combined preparation or pharmaceutical composition” for simultaneous, separate or sequential use, there is meant especially a “kit of parts” in the sense that the components nateglinide or repaglinide, respectively, and at least one further antidiabetic compound as mentioned above can be dosed independently or by use of different fixed combinations with distinguished amounts of the components, i.e. at different time points or simultaneously. The parts of the kit of parts can then e.g. be administered chronologically staggered, that is at different time points and with equal or different time intervals for any part of the kit of parts. Preferably, the time intervals are chosen such that the effect on the treated disease or condition in the combined use of the parts is larger than the effect which would be obtained by use of only any one of the components. Preferably, there is at least one beneficial effect, e.g. a mutual enhancing of the effect of the active ingredients, additional advantageous effects, less side-effects, a combined therapeutical effect in a non-effective dosage of one or each of the active ingredients, and especially a synergism, e.g. a more than additive effect, between nateglinide or repaglinide, respectively, and the at least one further antidiabetic compound as mentioned above.

Repaglinide is (S)-2-ethoxy-4-{2-[[3-methyl-1-[2-(1-piperidinyl)phenyl]butyl]amino]-2-oxoethyl}benzoic acid. Repaglinide is disclosed in EP 0 147 850 A2, in particular Example 11 on page 61, and EP 0 207 331 A1. It can be administered in the form as it is marketed e.g. under the trademark NovoNorm™.

Nateglinide is disclosed in EP 196222 and EP 526171. The term nateglinide as used herein comprises crystal modifications (polymorphs) such as those disclosed in EP 0526171 B1 or U.S. Pat. No. 5,488,510, respectively, the subject matter of which is incorporated by reference to this application, especially the subject matter of claims 8 to 10 as well as the corresponding references to the B-type crystal modification. Preferably, in the present invention the B- or H-type, more preferably the H-type, is used. Nateglinide can be administered in the form as it is marketed e.g. under the trademark STARLIX™.

The term “insulin signalling pathway modulators” as defined herein relates in particular to inhibitors of PTPase, antidiabetic non-small molecule mimetic compounds and inhibitors of GFAT.

Examples of “inhibitors of PTPase” include, but are not limited to those disclosed in U.S. Pat. No. 6,057,316, U.S. Pat. No. 6,001,867, WO 99/58518, WO 99/58522, WO 99/46268, WO 99/46267, WO 99/46244, WO 99/46237, WO 99/46236, WO 99/15529 and by Poucheret et al in Mol. Cell Biochem. 1998, 188, 73-80.

The term “antidiabetic non-small molecule mimetic compounds” as defined herein means compounds as disclosed in Science 1999, 284; 974-97, especially L-783,281, and WO 99/58127, especially CLX-901.

Examples of “inhibitors of GFAT” include, but are not limited to those disclosed in Mol. Cell. Endocrinol. 1997, 135(1), 67-77.

The term “compounds influencing a dysregulated hepatic glucose production” as defined herein relates in particular to inhibitors of glucose-6-phosphatase (G6Pase), inhibitors of fructose-1,6-bisphosphatase (F-1,6-BPase), inhibitors of glycogen phosphorylase (GP), glucagon receptor antagonists and inhibitors of phosphoenolpyruvate carboxykinase (PEPCK).

The term “inhibitors of G6Pase” used herein means a compound or composition which reduces or inhibits hepatic gluconeogenesis by decreasing or inhibiting the activity of G6Pase. Examples of such compounds are disclosed in WO 00/14090, WO 99/40062, WO 98/40385, EP682024 and Diabetes 1998, 47, 1630-1636.

The term “inhibitors of F-1,6-BPase” used herein means a compound or composition which reduces or inhibits hepatic gluconeogenesis by decreasing or inhibiting the activity of F-1,6-BPase. Examples of such compounds are disclosed in WO 00/14095, WO 99/47549, WO 98/39344, WO 98/39343 and WO 98/39342.

The term “inhibitors of GP” as used herein means a compound or composition which reduces or inhibits hepatic glycogenolysis by decreasing or inhibiting the activity of GP. Examples of such compounds are disclosed in EP 978279, U.S. Pat. No. 5,998,463, WO 99/26659, EP 846464, WO 97/31901, WO 96/39384, WO9639385 and in particular CP-91149 as described in Proc. Nalt. Acad Sci USA 1998, 95, 1776-1781.

The term “glucagon receptor antagonists” as used herein relates in particular to the compounds described in WO 98/04528, especially BAY27-9955, and those described in Bioorg Med. Chem. Left 1992, 2, 915-918, especially CP-99,711, J. Med. Chem. 1998, 41, 5150-5157, especially NNC 92-1687, and J. Biol Chem. 1999, 274; 8694-8697, especially L-168,049 and compounds disclosed in U.S. Pat. No. 5,880,139, WO 99/01423, U.S. Pat. No. 5,776,954, WO 98/22109, WO 98/22108, WO 98/21957 and WO 97/16442.

The term “inhibitors of PEPCK” used herein means a compound or composition which reduces or inhibits hepatic gluconeogenesis by decreasing or inhibiting the activity of PEPCK. Examples of such compounds are disclosed in U.S. Pat. No. 6,030,837 and Mol. Biol. Diabetes 1994, 2, 283-99.

The term “PDHK inhibitors” as used herein means inhibitors of pyruvate dehydrogenase kinase and include, but are not limited to, those compounds disclosed by Aicher et al in J. Med. Chem. 42 (1999) 2741-2746.

Examples of “inhibitors of gastric emptying” other than GLP-1 include, but are not limited to those disclosed in J. Clin. Endocrinol. Metab. 2000, 85(3), 1043-1048, especially CCK-8, and in Diabetes Care 1998; 21; 897-893, especially Amylin and analogs thereof, e.g. Pramlintide. Amylin is also described e.g. by O. G. Kolterman et al. in Diabetologia 39, 1996, 492-499.

Insulin is available from different providers under different tradenames, e.g. Berlinsulin© (Berlin-Chemie), Huminsulin© (Ell Lilly), Insulin Actrapid© (Novo Nordisk) or Insuman© (Aventis).

Examples of “inhibitors of GSK-3” include, but are not limited to those disclosed in WO 00/21927 and WO 97/41854.

By “RXR agonist” is meant a compound or composition which when combined with RXR homodimers or heterodimers increases the transcriptional regulation activity of RXR, as measured by an assay known to one skilled in the art, including, but not limited to, the “co-transfection” or “cis-trans” assays described or disclosed in U.S. Pat. Nos. 4,981,784, 5,071,773, 5,298,429, 5,506,102, WO 89/05355, WO 91/06677, WO92/05447, WO93/11235, WO95/18380, PCT/US93/04399, PCT/US94/03795 and CA 2,034,220, which are incorporated by reference herein. It includes, but is not limited to, compounds that preferentially activate RXR over RAR (i.e. RXR specific agonists), and compounds that activate both RXR and RAR (i.e. pan agonists). It also includes compounds that activate RXR in a certain cellular context but not others (i.e. partial agonists). Compounds disclosed or described in the following articles, patents and patent applications which have RXR agonist activity are incorporated by reference herein: U.S. Pat Nos. 5,399,586 and 5,466,861, WO96/05165, PCT/US95/16842, PCT/US95/16695, PCT/US93/10094, WO94/15901, PCT/US92/11214, WO093/11755, PCT/US93/10166, PCT/US93/10204, WO94/15902, PCT/US93/03944, WO93/21146, provisional applications 60,004,897 and 60,009,884, Boehm, et al. J. Med. Chem. 38(16):3146-3155, 1994, Boehm, et al. J. Med. Chem. 37(18):2930-2941, 1994, Antras et al., J. Biol. Chem. 266:1157-1161 (1991), Salazar-Olivo et al., Biochem. Biophys. Res. Commun. 204:157-263 (1994) and Safanova, Mol. Cell. Endocrin. 104:201-211 (1994). RXR specific agonists include, but are not limited to, LG 100268 (i.e. 2-[1-(3,5,5,8,8-pentamethyl-5,6,7,8-tetrahydro-2-naphthyl-cyclopropyl]-pyridine-5-carboxylic acid) and LGD 1069 (i.e. 4-[(3,5,5,8,8-pentamethyl-5,6,7,8-tetrahydro-2-naphthyl)-2-carbonyl]-benzoic acid), and analogs, derivatives and pharmaceutically acceptable salts thereof. The structures and syntheses of LG 100268 and LGD 1069 are disclosed in Boehm, et al. J. Med. Chem. 38(16):3146-3155, 1994, incorporated by reference herein. Pan agonists include, but are not limited to, ALRT 1057 (i.e. 9-cis retinoic acid), and analogs, derivatives and pharmaceutically acceptable salts thereof.

Examples of “agonists of Beta-3 AR” include, but are not limited to CL-316,243 (Lederle Laboratories) and those disclosed in WO 99/29672, WO 98/32753, WO 98/20005, WO 98/09625, WO 97/46556, WO 97/37646 and U.S. Pat. No. 5,705,515.

The term “agonists of UCPs” used herein means agonists of UCP-1, preferably UCP-2 and even more preferably UCP-3. UCPs are disclosed in Vidal-Puig et al., Biochem. Biophys. Res. Commun., Vol. 235(1) pp. 79-82 (1997). Such agonists are a compound or composition which increases the activity of UCPs.

“Non-glitazone type PPARγ agonists” are especially N-(2-benzoylphenyl)-L-tyrosine analogues, e.g. GI-262570, and JTT501.

The term “dual PPARγ/PPARα agonists” as used herein means compounds which are at the same time PPARγ and PPARα agonists. Preferred dual PPARγ/PPARα agonists are especially those ω-[(oxoquinazolinylalkoxy)phenyl]alkanoates and analogs thereof in the form of addition salts or esters, very especially the compound of formula (II)

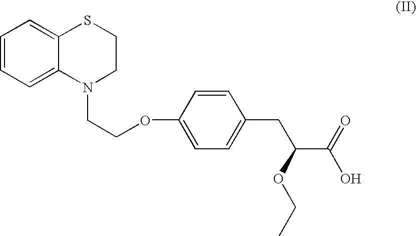

which is described in WO 99/20614 and the compound NC-2100 described by Fukui in Diabetes 2000, 49(5), 759-767. The compound of formula (II), 3[4-[2-(2,3-dihydro-1,4-benzothiazin-4-yl)ethoxy]phenyl]-2-ethoxypropionic acid, is also alternatively designated DRF 554158 and DRF 4158, respectively.

Preferably, the “antidiabetic vanadium containing compound” is a physiologically tolerable vanadium complex of a bidentate monoprotic chelant, wherein said chelant is an α-hydroxypyrone or α-hydroxypyridinone, especially those disclosed in the Examples of U.S. Pat. No. 5,866,563, of which the working examples are hereby incorporated by reference, or a pharmaceutically acceptable salt thereof.

The term “incretin hormones” as used herein relates in particular to glucagon-like peptide-1 (GLP-1) or GLP-1 agonists. GLP-1 is a insulinotropic proteine which was described, e.g., by W. E. Schmidt et al. in Diabetologia 28, 1985, 704-707 and in U.S. Pat. No. 5,705,483. The term “GLP-1 agonists” used herein means variants and analogs of GLP-1(7-36)NH₂ which are disclosed in particular in U.S. Pat. No. 5,120,712, U.S. Pat. No. 5,118,666, U.S. Pat. No. 5,512,549, WO 91/11457 and by C. Orskov et al in J. Biol. Chem. 264 (1989) 12826. The term “GLP-1 agonists” comprises especially compounds like GLP-1(7-37), in which compound the carboxy-terminal amide functionality of Arg³⁶ is displaced with Gly at the 37^{th} position of the GLP-1(7-36)NH₂ molecule and variants and analogs thereof including GLN⁹-GLP-1(7-37), D-GLN⁹-GLP-1(7-37), acetyl LYS⁹-GLP-1(7-37), LYS¹⁸-GLP-1(7-37) and, in particular, GLP-1(7-37)OH, VAL⁸-GLP-1(7-37), GLY⁸-GLP-1(7-37), THR⁸-GLP-1(7-37), MET⁸-GLP-1(7-37) and 4-imidazopropionyl-GLP-1. Special preference is also given to the GLP agonist analog exendin-4, described by Greig et al in Diabetologia 1999, 42, 45-50.

The term “β-cell imidazoline receptor antagonists” as used herein means compounds as those described in WO 00/78726 and by Wang et al in J. Pharmacol. Exp. Ther. 1996; 278; 82-89, e.g. PMS 812.

Miglitol is (2R, 3R, 4R, 5S)-1-(2-hydroxyethyl)-2-(hydroxymethyl)-3,4,5-piperidinetriol and is described in U.S. Pat. No. 4,639,436. The 1-deoxynojirimycin derivative miglitol can be administered in the form as it is marketed e.g. under the trademark DIASTABOL 50™.

Examples of “α₂-adrenergic antagonists” include, but are not limited to midaglizole described in Diabetes 36, 1987, 216-220.

The insulin signalling pathway modulators, compounds influencing a dysregulated hepatic glucose production, pyruvate dehydrogenase kinase (PDHK) inhibitors, inhibitors of gastric emptying, inhibitors of GSK-3, retinoid X receptor (RXR) agonists, agonists of Beta-3 AR, agonists of UCPs, non-glitazone type PPARγ agonists, dual PPARγ/PPARα agonists, antidiabetic vanadium containing compounds, incretin hormones, β-cell imidazoline receptor antagonists, miglitol, and α₂-adrenergic antagonists are in each case generically and specifically disclosed in the documents cited above, in each case in particular in the compound claims and the final products of the working examples, the subject-matter of the final products, the pharmaceutical preparations and the claims are hereby incorporated into the present application by reference to these publications. Comprised are likewise the corresponding stereoisomers as well as the corresponding crystal modifications, e.g. solvates and polymorphs, which are disclosed therein.

The structure of the active agents identified by code nos., generic or trade names may be taken from the actual edition of the standard compendium “The Merck Index” or from databases, e.g. Patents International (e.g. IMS World Publications). The corresponding content thereof is hereby incorporated by reference.

Any person skilled in the art is fully enabled to identify the active agents and, based on these references, likewise enabled to manufacture and test the pharmaceutical indications and properties in standard test models, both in vitro and in vivo.

It will be understood that in the discussion of methods, references to the active ingredients are meant to also include the pharmaceutically acceptable salts. If these active ingredients have, for example, at least one basic center, they can form acid addition salts. Corresponding acid addition salts can also be formed having, if desired, an additionally present basic center. The active ingredients having an acid group (for example COOH) can also form salts with bases. The active ingredient or a pharmaceutically acceptable salt thereof may also be used in form of a hydrate or include other solvents used for crystallization.

The combination which comprises nateglinide of formula (I) or repaglinide and at least one further antidiabetic compound selected from the group consisting of insulin signalling pathway modulators, compounds influencing a dysregulated hepatic glucose production, pyruvate dehydrogenase kinase (PDHK) inhibitors, inhibitors of gastric emptying, insulin, inhibitors of GSK-3, retinoid X receptor (RXR) agonists, agonists of Beta-3 AR, agonists of UCPs, non-glitazone type PPARγ agonists, dual PPARγ/PPARα agonists, antidiabetic vanadium containing compounds, incretin hormones, β-cell imidazoline receptor antagonists, miglitol, and α₂-adrenergic antagonists, in which the active ingredients are present in each case in free form or in the form of a pharmaceutically acceptable salt, if at least one salt-forming group is present, will be referred to hereinafter as a COMBINATION OF THE INVENTION.

The term “prevention” means prophylactic administration of the combination to healthy patients to prevent the outbreak of the diseases and conditions mentioned herein. Moreover, the term “prevention” means prophylactic administration of such combination to patients being in a pre-stage of the disease, especially diabetes, to be treated.

The term “delay of progression” used herein means administration of the combination to patients being in a pre-stage of the disease, especially diabetes, to be treated in which patients a pre-form of the corresponding disease is diagnosed.

“Diseases and conditions associated with diabetes mellitus” as defined in this application comprise, but are not limited to hyperglycemia, hyperinsulinaemia, hyperlipidaemia, insulin resistance, impaired glucose metabolism, obesity, diabetic retinopathy, macular degeneration, cataracts, diabetic nephropathy, glomerulosclerosis, diabetic neuropathy, erectile dysfunction, premenstrual syndrome, vascular restenosis, ulcerative colitis, coronary heart disease, hypertension, angina pectoris, myocardial infarction, stroke, skin and connective tissue disorders, foot ulcerations, metabolic acidosis, arthritis, osteoporosis and in particular conditions of impaired glucose tolerance.

The nature of diabetes and related diseases or conditions is multifactorial. Under certain circumstances, drugs with different mechanisms of action may be combined. However, just considering any combination of drugs having different mode of action but acting in the similar field does not necessarily lead to combinations with advantageous effects.

All the more surprising is the experimental finding that the combined administration of a COMBINATION OF THE INVENTION, results in a beneficial, especially a synergistic, therapeutic effect and/or in additional benefits resulting from combined treatment such as a surprising prolongation of efficacy, a broader variety of therapeutic treatment and surprising beneficial effects on diseases and conditions associated with diabetes, e.g. less gain of weight, compared to a monotherapy applying only one of the pharmaceutically active ingredients used in the COMBINATION OF THE INVENTION.

It can be shown by established test models and especially those test models described herein that the COMBINATION OF THE INVENTION results in a more effective prevention or preferably treatment of diseases, especially metabolic disorders, and in particular type 2 diabetes mellitus and diseases and conditions associated with diabetes mellitus.

If taken simultaneously, this results not only in a further enhanced beneficial, especially a synergistic, therapeutic effect, but also in additional benefits resulting from the simultaneous treatment such as a surprising prolongation of efficacy, a broader variety of therapeutic treatment and surprising beneficial effects, e.g. less increase of weight, on diseases and conditions associated with diabetes mellitus, for the combinations as described herein. Moreover, for a human patient, especially for elderly people, it is more convenient and easier to remember to take two tablets at the same time, e.g. before a meal, than staggered in time, i.e. according to a more complicated treatment schedule. Also for this reason, most preferably, both active ingredients are administered as a fixed combination, as applied in a unit dosage form, e.g., in such a case as a single tablet, in all cases described herein. Taking a single tablet is easier to handle than taking two tablets at the same time. Furthermore, the packaging can be accomplished with less effort. Accordingly, the present invention relates in particular to a fixed combination comprising a COMBINATION OF THE INVENTION.

The person skilled in the pertinent art is fully enabled to select a relevant animal test model to prove the hereinbefore and hereinafter indicated therapeutic indications and beneficial effects. The pharmacological activity may, for example, be demonstrated following essentially an in-vivo test procedure in mice or in a clinical study as described hereinafter.

### In-vivo Test in Mice for Blood Glucose Control

ICR-CDI mice (male, five weeks old, body weight about 20 g) are abstained from food for 18 hours, and then used as test subjects. A COMBINATION OF THE INVENTION and the active ingredients alone are suspended in 0.5% CMC-0.14 M sodium chloride buffer solution (pH 7.4). The solutions thus obtained are administered orally in fixed volume amounts to the test subjects. After predetermined time, the percentage decrease of the blood glucose against the control group is determined.

Clinical Double-Blind, Randomized, Parallel-Group Study in Subjects with Non-Insulin Dependent Diabetes Mellitus (Type 2 Diabetes Mellitus) Inadequately Controlled on Diet Alone

These studies prove, e.g., the synergism of the COMBINATION OF THE INVENTION. The beneficial effects on diseases and conditions associated with diabetes as defined in this application can be determined directly through the results of these studies or by changes in the study designs which are known as such to a person skilled in the art.

The studies are, in particular, suitable to assess the effects of monotherapy with nateglinide (I) or repaglinide and the other active ingredients mentioned herein or a COMBINATION OF THE INVENTION on glycemic control. Subjects with a diagnosis of type 2 diabetes mellitus who have not achieved near normoglycemia (HbA_{1c} (glycosylated haemoglobin) <6.8%) on diet only are chosen for these trial. The effects on glycemic control are determined in these studies with the control achieved on placebo, all subjects continuing with the same diet as in the period before treatment Measures of glycemic control are validated surrogate endpoints for the treatment of diabetes. HbA_{1c} is the single most reliable measurement for assessing glycemic control (D. Goldstein et al, Tests of Glycemia in Diabetes; Diabetes Care 1995, 18(6), 896-909) and is the primary response variable in this study. Since glycosylation of hemoglobin is determined by the glucose concentration at the time each red blood cell is made, HbA_{1c} provides an estimate of mean blood glucose for the previous three months.

Before starting with the double-blind treatment for 24 weeks, the subjects are administered for four weeks nateglinide or repaglinide matching placebos before breakfast, lunch and dinner, and a placebo matching the combination partner, in particular selected from CLX-901, BAY27-9955, CP-99,711, amylin, LG 100268, LGD 1069, ALRT 1057, CL-316,243, GI-262570, JTT501, GLP-1, GLP-1(7-37)OH, VAL⁸-GLP-1(7-37), GLY⁸-GLP-1(7-37), THR⁸-GLP-1(7-37), MET⁸-GLP-1(7-37), 4-imidazopropionyl-GLP-1, PMS 812 and miglitol, administered later on with breakfast, lunch and dinner or according to the preferred treatment schedule for the respective combination partner (period I). The subjects are then separated into four treatment groups for the 24-week double-blind study (period II) as depicted in Table 1. Approximately 50 to 200 subjects are randomized per treatment group. The total study duration including the run-in period for each subject can be, e.g., 28 weeks. Statistical analysis can be carried out by methods known in the art.

TABLE 1
Examples for a Combination comprising
Nateglinide or Repaglinide

nateglinide (I) 120 mg* or repaglinide 1 mg* + combination partner
placebo
repaglinide* or nateglinide (I) placebo* + combination partner
nateglinide (I) 120 mg* or repaglinide 1 mg* + combination partner
repaglinide* or nateglinide (I) placebo* + combination partner placebo
*administered before breakfast, lunch, and dinner

Nateglinide tablets contain either 120 mg or matching placebo. Repaglinide 1 mg tablets and tablets containing the combination partners can, e.g., be purchased commercially and overencapsulated to match the corresponding placebo capsules.

For example, the following procedure can be followed in order to take blood samples: The subject is advised not to take the morning dose of study medication or eat breakfast on the day of a scheduled study visit. The morning dose is administered by site personnel after the collection of all fasting laboratory samples and completion of all study procedures. Visits are scheduled to be performed at 2 week intervals during period I, and 4 to 8 week intervals during period II. Subjects have fasted for at least 7 hours at the time of each visit. All blood samples for laboratory evaluations are drawn between 7:00 AM and 10:00 AM. All tests are conducted in accordance with GLP (Good Laboratory Practice) principles following procedures known in the art.

HbA_{1c} is measured by High Performance Liquid Chromatography (HPLC) using the ion-exchange method on a Bio-Rad Diamat analyzer. A back-up affinity method are used if hemoglobin variants or hemoglobin degradation peaks are observed. Further parameters to be determined are fasting plasma glucose (FPG), fasting lipids (total, HDL (high density lipoprotein)- and LDL (low density lipoprotein)-cholesterol, and triglycerides) and body weight. FPG will be measured using the hexokinase method and LDL-cholesterol will be calculated using the Friedewald formula if triglycerides are <400 mg/dL (4.5 mmol/l).

Various parameters of the study described above can be modified, e.g. in order to optimize the dosage for special diseases or indications mentioned herein, to cope with tolerability problems during the study or to obtain similar or identical results with less efforts. For example, a different subject population can be involved in such a clinical trial, e.g. subjects with a diagnosis of type 2 diabetes mellitus who have achieved near normoglycemia (HbA_{1c} <6.8%) on diet alone, subjects with diseases other than diabetes mellitus, e.g. other metabolic disorders, or subjects selected by other criteria, such as age or sex; the subject number can be decreased, e.g. to a number of between 70 and 150, especially 100 or 120, subjects per treatment group; treatment groups can be deleted, i.e. for example to carry out a study with a comparison of the combination of nateglinide and an antidiabetic phenylacetic acid versus the single antidiabetic phenylacetic acid only; the term of the placebo run-in period (period I) can be changed, i.e. it can be extended, shortened or deleted, the visit schedule can be extended, e.g. to every 10, 12 or 14 weeks; the visit instructions can be changed, e.g. the instruction that blood samples for laboratory evaluations have to be drawn between 7:00 AM and 10:00 AM; HbA_{1c} can be determined by other means; or one or more of the parameters to be determined during the study mentioned above, e.g. (FPG) or fasting lipids, can be deleted or. the determination of additional parameters (see below) can be added.

Additional parameters can be determined in the course of the study, e.g. by additional tests. Such additional tests can comprise the analysis of body liquids in order to determine amounts or numbers for parameters such as those listed below and can serve e.g. the purpose of determining the tolerability of the administered active ingredients: determination of hematocrit and hemogloblin, platelet count, erythrocyte count, total and differential leukocyte count (basophils, eosinophils, lymphocytes, monocytes, segmented neutrophils and total neutrophils); determination of albumin, alkaline phosphatase, alanine amino transferase (serum glutamic pyruvic transaminase), aspartate amino transferase (serum glutamic oxaloacetic transaminase), blood urea nitrogen or urea, bicarbonate, calcium, chloride, total creatine phosphokinase (CPK), creatine phosphokinase muscle-brain fraction isoenzyme (if CPK is elevated), direct bilirubin, creatinine, γ-glutamyl transferase, lactate dehydrogenase, potassium, sodium, total bilirubin, total protein and uric acid in the blood; determination of bilirubin, glucose, ketones, pH, protein, and specific gravity in the subjects urine; determination of body weight, blood pressure (systolic and diastolic, after 3 minutes sitting) and radial pulse (after 3 minutes sitting).

The combined administration of the COMBINATION OF THE INVENTION results in a beneficial, especially a synergistic, therapeutic effect, especially on type 2 diabetes, and/or in additional benefits such as a decrease of diabetes-related mortality, a surprising prolongation of efficacy of the drug (such delaying the eventual need for insulin), a broader variety of therapeutic treatment, maintaining the target blood glucose level in type 2 diabetes patients, providing a good initial blood glucose control in type 2 diabetes patients, only modest changes in fasting plasma glucose level, and further surprising beneficial effects, comprising e.g. less or no gain of body weight, a decrease of gastrointestinal side effects or an improved safety profile, compared to a monotherapy applying only one of the active ingredients used in the COMBINATION OF THE INVENTION. In particular, the further surprising beneficial effects can also be observed during the treatment of metabolic disorders other than type 2 diabetes and during the treatment of diseases and conditions associated with type 2 diabetes. Further benefits are, e.g., that lower doses of the individual drugs to be combined according to the present invention can be used to reduce the dosage, for example, that the dosages need not only often be smaller but are also applied less frequently, or can be used in order to diminish the incidence of side effects (e.g. anaemia, oedema, headache).

Furthermore, in a number of combinations as disclosed herein the side-effects observed with one of the active ingredients surprisingly do not accumulate on application of the COMBINATION OF THE INVENTION.

The beneficial therapeutic effects, additional benefits and also the surprising beneficial effects are observed especially in human subjects suffering from a more severe form of type 2 diabetes, i.e. human subjects having an elevated HbA_{1c} value at baseline of greater 8% and more particular in human subjects having a HbA_{1c} value at baseline of greater than 9.5%, before treatment with the combinations described herein. If nateglinide is administered to such human patients, it is applied preferably in a dose of between 90 and 200 mg, more preferably between 100 and 150 mg, for example 120 mg, nateglinide per meal as part of the COMBINATION OF THE INVENTION given to them.

Furthermore, the beneficial therapeutic effects, additional benefits and also the surprising beneficial effects are observed especially in human subjects having a body mass index (BMI) of 20 to 35 kg/m², in particular a BMI of 27 to 35 kg/m², and even more enhanced in human subjects with a BMI of 30 to 35 kg/m². Human subjects having a BMI greater 30 kg/m² are defined to be clinically obese.

Additionally, the beneficial therapeutic effects, additional benefits and also the surprising beneficial effects are observed especially in patients poorly controlled by monotherapy with one of the components of the COMBINATION OF THE INVENTION.

Furthermore, the invention relates to a combination which comprises nateglinide and at least one further antidiabetic compound selected from the group consisting of insulin signalling pathway modulators, compounds influencing a dysregulated hepatic glucose production, pyruvate dehydrogenase kinase (PDHK) inhibitors, inhibitors of gastric emptying, insulin, inhibitors of GSK-3, retinoid X receptor (RXR) agonists, agonists of Beta-3 AR, agonists of UCPs, non-glitazone type PPARγ agonists, dual PPARγ/PPARα agonists, antidiabetic vanadium containing compounds, incretin hormones, β-cell imidazoline receptor antagonists, miglitol, and α₂-adrenergic antagonists; and at least one further pharmaceutically active compound selected from the group consisting of antidiabetic thiazolidinediones (glitazones), sulphonyl urea derivatives, metformin, acarbose or the pharmaceutically acceptable salts of such compounds where possible. Preferably, the at least one further pharmaceutically active compound selected from the group above is metformin or a glitazone or the pharmaceutically acceptable salts of such compounds where possible.

The term “glitazone” as used herein means in particular a compound selected from (S)-((3,4-dihydro-2-(phenyl-methyl)-2H-1-benzopyran-6yl)methyl-thiazolidine-2,4-dione (englitazone, EP 0 207 605 B1), 5-{[4-(3-(5-methyl-2-phenyl-4-oxazolyl)-1-oxopropyl)-phenyl]methyl}-thiazolidine-2,4-dione (darglitazone, EP 0 332 332), 5-{[4-(1-methyl-cyclohexyl)methoxy)-phenyl]methyl}-thiazolidine-2,4-dione (ciglitazone, U.S. Pat. No. 4,287,200), 5-{[4-(2-(1-indolyl)ethoxy)phenyl]methyl}-thiazolidine-2,4-dione (DRF2189), 5-{4-[2-(5-methyl-2-phenyl-4-oxazolyl)-ethoxy)]benzyl}-thiazolidine-2,4-dione (BM-13.1246), 5-(2-naphthylsulfonyl)-thiazolidine-2,4dione (AY-31637, U.S. Pat. No. 4,997,948), bis{4-[(2,4-dioxo-5-thiazolidinyl)methyl]phenyl}methane (YM268), 5-{4-[2-(5-methyl-2-phenyl-4-oxazolyl)-2-hydroxyethoxy]benzyl}-thiazolidine-2,4-dione (AD5075), 5-[4-(1-phenyl-1-cyclopropanecarbonylamino)-benzyl]-thiazolidine-2,4-dione (DN-108) 5-{[4-(2-(2,3-dihydroindol-1-yl)ethoxy)phenyl]methyl}-thiazolidine-2,4-dione, 5-[3-(4-chloro-phenyl])-2-propynyl]-5-phenylsulfonyl)thiazolidine-2,4-dione, 5-[3-(4-chlorophenyl])-2-propynyl]-5-(4-fluorophenyl-sulfonyl)thiazolidine-2,4-dione, 5-{[4-(2-(methyl-2-pyridinyl-amino)-ethoxy)phenyl]methyl}-thiazolidine-2,4-dione (rosiglitazone, EP 0 306 228 A1), 5-{[4-(2-(5-ethyl-2-pyridyl)ethoxy)phenyl]-methyl}thiazolidine-2,4-dione (pioglitazone, EP 0 193 256 A1), 5-{[4-((3,4-dihydro-6-hydroxy-2,5,7,8-tetramethyl-2H-1-benzopyran-2-yl)methoxy)-phenyl]-methyl}-thiazolidine-2,4-dione (troglitazone, EP 0 139 421), 5-[6-(2-fluoro-benzyloxy)naphthalen-2-ylmethyl]-thiazolidine-2,4dione (MCC555, EP 0 604 983 B1), 5-{[2-(2-naphthyl)-benzoxazol-5-yl]-methyl}thiazolidine-2,4-dione (T-174) and 5-(2,4-dioxothiazolidin-5-ylmethyl)-2-methoxy-N-(4-trifluoromethyl-benzyl)benzamide (KRP297, JP 10087641-A). The compounds are in each case generically and specifically disclosed in the documents cited in brackets beyond each substance, in each case in particular in the compound claims and the final products of the working examples, the subject-matter of the final products, the pharmaceutical preparations and the claims are hereby incorporated into the present application by reference to these publications. Comprised are likewise the corresponding stereoisomers as well as the corresponding crystal modifications, e.g. solvates and polymorphs, which are disclosed therein.

MCC555 can be formulated as disclosed on page 49, lines 30 to 45, of EP 0 604 983 B1; englitazone as disclosed from page 6, line 52, to page 7, line 6, or analogous to Examples 27 or 28 on page 24 of EP 0 207 605 B1; and darglitazone and BM-13.1246 can be formulated as disclosed on page 8, line 42 to line 54 of EP 0 332 332 B1. AY-31637 can be administered as disclosed in column 4, lines 32 to 51 of U.S. Pat. No. 4,997,948 and rosiglitazone as disclosed on page 9, lines 32 to 40 of EP 0 306 228 A1, the latter preferably as its maleate salt. Corresponding to the needs of the single patient and under the proviso that it is intended by a physician to administer the combinations, e.g. the pharmaceutical compositions, in separate tablets, ft is possible to administer the antidiabetics as launched, e.g. rosiglitazone in the form as it is launched under the trademark AVANDIA™. Troglitazone can be administered in the form as it is launched under the trademarks ReZulin™, PRELAY™, ROMOZIN™ (in the United Kingdom) or NOSCAL™ (in Japan). Pioglitazone can be administered as disclosed in Example 2 of EP 0 193 256 A1, preferably in the form of the monohydrochloride salt or in the form as launched under the trademark ACTOS™. Ciglitazone can, for example, be formulated as disclosed in Example 13 of U.S. Pat. No. 4,287,200.

Preferably, the glitazone is selected from the group consisting of rosiglitazone, pioglitazone and troglitazone, or a pharmaceutically acceptable salt thereof.

The sulphonyl urea derivative is, for example, gilsoxepid, glyburide, glibenclamide, acetohexamide, chloropropamide, glibomuride, tolbutamide, tolazamide, glipizide, carbutamide, gliquidone, glyhexamide, phenbutamide or tolcyclamide; and preferably glimepiride or gliclazide. Tolbutamide, glibenclamide, gliclazide, gribomuride, gliquidone, glisoxepid and glimepiride can be administered e.g. in the form as they are marketed under the trademarks RASTINON HOECHST™, AZUGLUCON™, DIAMICRO™, GLUBORID™, GLURENORM™, PRO-DIABAN™ and AMARYL™, respectively.

Acarbose (O-4,6-dideoxy-4-{[1S,4R,5S,6S]-4,5,6-trihydroxy-3-(hydroxymethyl)-2-cyclohexen-1-yl]-amino}-α-D-glucopyranosyl-(1→4)-O-α-D-glucopyranosyl-(1→4)-D-glucopyranose) was described, for example, in U.S. Pat. No. 4,062,950. Acarbose can be administered in the form as it is marketed e.g. under the trademark GLUCOBAY™

The preparation of metformin (dimethyldiguanide) and its hydrochloride salt is state of the art and was disclosed by Emil A. Werner and James Bell, J. Chem. Soc. 121, 1922, 1790-1794. The drug is further described, e.g., in U.S. Pat. No. 3,174,901. If the drug metformin shall be administered in a separate pharmaceutical composition, it can be administered in the form as it is launched e.g. under the trademark DIABETOSAN™. If the drug metformin shall be administered in a separate pharmaceutical composition in the form of its hydrochloride salt, the metformin hydrochloride salt can be administered in the form as it is launched e.g. under the trademarks DIABETASE 500™, DIABETASE 850™ or GLUCOPHAGE S™.

It is one objective of this invention to provide a pharmaceutical composition comprising a amount, which is jointly therapeutically effective against metabolic disorders, more especially diabetes and in particular type 2 diabetes mellitus or a disease or condition associated with diabetes, of the COMBINATION OF THE INVENTION and at least one pharmaceutically acceptable carrier. In this composition, the active ingredients can be administered together, one after the other or separately in one combined unit dosage form or in two separate unit dosage forms. The unit dosage form may also be a fixed combination.

The pharmaceutical compositions according to the invention can be prepared in a manner known per se and are those suitable for enteral, such as oral or rectal, and parenteral administration to mammals (warm-blooded animals), including man, comprising a therapeutically effective amount of the pharmacologically active compound, alone or in combination with one or more pharmaceutically acceptable carries, especially suitable for enteral or parenteral application.

The novel pharmaceutical preparations contain, for example, from about 10% to about 100%, preferably 80%, preferably from about 20% to about 60%, of the active ingredient.

Pharmaceutical preparations for the combination therapy that may be used for enteral or parenteral administration are, for example, those in unit dose forms, such as sugar-coated tablets, tablets, capsules or suppositories, and furthermore ampoules. If not indicated otherwise, these are prepared in a manner known per se, for example by means of conventional mixing, granulating, sugar-coating, dissolving or lyophilizing processes. Thus, pharmaceutical preparations for oral use can be obtained by combining the active ingredient with solid carriers, if desired granulating a mixture obtained, and processing the mixture or granules, if desired or necessary, after addition of suitable excipients to give tablets or sugar-coated tablet cores.

It will be appreciated that the unit content of active ingredient or ingredients contained in an individual dose of each dosage form need not in itself constitute an effective amount since the necessary effective amount can be reached by administration of a plurality of dosage units.

In particular, a therapeutically effective amount of each of the components of the COMBINATION OF THE INVENTION may be administered simultaneously or sequentially and in any order, and the components may be administered separately or as a fixed combination. For example, the method of prevention, delay of progression or treatment of according to the invention may comprise (i) administration of nateglinide or repaglinide, respectively, in free or pharmaceutically acceptable salt form and (ii) administration of combination partner in free or pharmaceutically acceptable salt form, simultaneously or sequentially in any order, in jointly therapeutically effective amounts, preferably in synergistically effective amounts, e.g. in daily dosages corresponding to the ratios described herein. The individual components of the COMBINATION OF THE INVENTION can be administered separately at different times during the course of therapy or concurrently in divided or single combination forms. For example, in a two-component combination of, e.g., nateglinide or repaglinide, respectively, and GLP-1, treatment with nateglinide or repaglinide, respectively, can commence prior to, subsequent to or concurrent with commencement of treatment with GLP-1. Furthermore, the term administering also encompasses the use of prodrugs of any of the anti-diabetic drugs that convert in vivo to the selective anti-diabetic drug. The instant invention is therefore to be understood as embracing all such regimes of simultaneous or alternating treatment and the term “administering” is to be interpreted accordingly.

The preferred route of administration of the dosage forms of the present invention is orally or enterally. In practical use, the anti-diabetic drugs or combinations thereof can be combined as the active ingredients in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed or carriers, diluents, granulating agents, lubricants, binders, disintegrating agents and the like in the case of oral solid preparations such as, for example, powders, capsules and tablets, with the solid oral preparations being preferred over the liquid preparations. Because of their ease of administration, tablets and capsules represent the most advantageous oral dosage unit form in which case solid pharmaceutical carriers are obviously employed.

If the active ingredients are administered, the pharmaceutical composition, comprising solely nateglinide can be produced by a process that comprises granulating in the presence of water to form granules, drying the granules, and optionally screening the granules, for example, through a wire mesh screen. All of the ingredients of the composition may be added prior to or during the granulation. Alternatively, all or a portion of one or more of the ingredients may be added after the granulation step is complete. For example, all or a portion of anti-adherent (e.g., silica), all or a portion of lubricant (e.g., magnesium stearate) and/or all or a portion of disintegrant (e.g., croscarmellose or any salt thereof may be added after the granulation. In one aspect of the invention, all ingredients except the magnesium stearate and the colloidal silica are loaded into the granulator, then they are added later. The process of producing this composition, in particular pharmaceutical composition, may be performed without the need for a pulverization step. As used herein, the terms “pulverization” and “pulverize” refer to any process that involves the grinding or smashing cutting of particles to reduce the particles' size. The composition, in particular pharmaceutical composition, is capable of being produced without pulverizing the granules between the granulation step and the drying and/or compression step used to form the granules into a tablet.

A further aspect of the present invention is the use of a pharmaceutical composition comprising the COMBINATION OF THE INVENTION for the preparation of a medicament for the prevention, delay of progression or treatment of metabolic disorders, in particular of type 2 diabetes mellitus or a disease or condition associated with diabetes mellitus.

Further aspects of the present invention are oral dosage forms and pharmaceutical formulations (compositions) for administration to mammals suffering from or at risk for diseases having the characteristics of type 2 diabetes. It will be understood that any statistically significant attenuation in the disease symptoms of type 2 diabetes pursuant to the treatment of the present invention is within the scope of the invention.

The term “combination therapy” as used herein means that a COMBINATION OF THE INVENTION is used for the treatment, delay of progression or prevention of one of the diseases, especially metabolic disorders, mentioned herein.

In accordance with the combination therapies of the present invention there is further provided a method of prevention, delay of progression or treatment of and a pharmaceutical composition for the prevention, delay of progression or treatment of obesity and diabetes. The treatment involves administering to a patient in need of such treatment a pharmaceutical composition comprising a pharmaceutical carrier and a therapeutically effective amount of the COMBINATION OF THE INVENTION.

Furthermore, the invention relates to a pharmaceutical composition comprising the COMBINATION OF THE INVENTION for the prevention, delay of progression or treatment of hyperglycemia, hyperinsulinaemia, hyperlipidaemia, insulin resistance, impaired glucose metabolism, obesity, diabetic retinopathy, macular degeneration, cataracts, diabetic nephropathy, glomerulosclerosis, diabetic neuropathy, erectile dysfunction, premenstrual syndrome, vascular restenosis, ulcerative colitis, coronary heart disease, hypertension, angina pectoris, myocardial infarction, stroke, skin and connective tissue disorders, foot ulcerations, metabolic acidosis, arthritis, osteoporosis and in particular conditions of impaired glucose tolerance and, especially, type 2 diabetes.

A further aspect of the present invention is a method of treatment of a warm-blooded animal, especially a human, having metabolic disorders, in particular type 2 diabetes mellitus or a disease or condition associated with diabetes mellitus, comprising administering to the animal a COMBINATION OF THE INVENTION in an amount which is jointly therapeutically effective against metabolic disorders in which the active ingredients can also be present in the form of their pharmaceutically acceptable salts simultaneously or sequentially in any order, separately or in a fixed combination.

The invention relates also to a COMBINATION OF THE INVENTION for use in the prevention, delay of progression or treatment of diseases, the use of such combination for the preparation of a medicament for the prevention, delay of progression or treatment of metabolic disorders, and the use of such combination for the cosmetic treatment of a mammal in order to effect a cosmetically beneficial loss of body weight.

Furthermore, the invention relates to a method of improving the bodily appearance of a mammal, including man, especially man suffering from a metabolic disorder, in particular type 2 diabetes, which comprises orally administering to said mammal a COMBINATION OF THE INVENTION in a dosage effective to influence, e.g. to increase or decrease, the glucose metabolism, or to influence the body weight by other mechanisms, and repeating said dosage until a cosmetically beneficial loss of body weight has occurred. The COMBINATION OF THE INVENTION can also be used to prevent, for cosmetic reasons, a further increase in body weight in humans experiencing such an increase. Overweight is one of the risk factors for developing a metabolic disorder, in particular type 2 diabetes, and at the same time often the result of such a metabolic disorder, especially type 2 diabetes. Furthermore, a number of antidiabetics are known to cause weight gain. Hence, humans suffering from metabolic disorders, especially type 2 diabetes, are often faced with overweight. Therefore, the cosmetically beneficial loss of body weight can be effected especially in humans suffering from a metabolic disorder, such as type 2 diabetes. The COMBINATION OF THE INVENTION can also be used to replace or complement an antidiabetic drug taken by a human suffering from type 2 diabetes in order to prevent for cosmetic reasons a further increase of the body weight.

The invention relates in particular to a commercial package comprising jointly therapeutically effective amounts of COMBINATION OF THE INVENTION together with instructions for use thereof in the treatment of metabolic disorders, more especially diabetes, or a disease or condition associated with diabetes.

The effective dosage of each of the active ingredients employed in the combination therapy may vary depending on the particular compound or pharmaceutical composition employed, the mode of administration, the condition being treated, the severity of the condition being treated, the species of the warm-blooded animal, body weight, sex, diet and age. Thus, the dosage regimen utilizing the compounds of the present invention is selected in accordance with a variety of factors including the route of administration and the renal and hepatic function of the patient. A physician, clinician or veterinarian of ordinary skill can readily determine and prescribe the effective amount of the drug required to prevent, counter or arrest the progress of the condition. Optimal precision in achieving concentration of drug within the range that yields efficacy without toxicity requires a regimen based on the kinetics of the drug's availability to target sites. This involves a consideration of the distribution, equilibrium, and elimination of a drug. Hence, the dosage regimen, i.e. dose level and frequency of dosage, of any of the individual components of the COMBINATION OF THE INVENTION as described hereinafter may be adjusted to provide the optimal therapeutic response. Unless stated otherwise herein, the COMBINATION OF THE INVENTION is divided and administered from one to four times per day. Preferably, the COMBINATION OF THE INVENTION is taken together with or, more preferably, before every meal.

Nateglinide is preferably administered to the warm-blooded animal in a dosage in the range of about 120 to 1200, more preferably 360 to 800 mg/day, especially when the warm-blooded animal is a human of about 70 kg body weight.

If the the warm-blooded animal is a human the dosage of repaglinide is preferably in the range of about 0.25 to 100, more preferably about 0.5 to 16, and most preferably 1 to 8, mg/day, per adult patient.

If the the warm-blooded animal is a human of about 70 kg body weight the dosages of the at least one further pharmaceutically active compounds are preferably the following:

TABLE 2

| pharmaceutically active | | |
|---|---|---|
| compound | preferred dosage | most preferred dosage |
| acarbose | about 50 to 600 mg/day | about 150 to 300 mg/day |
| AD-5075 | about 0.1 to 2500 mg/day | about 1 to 1000 mg/day |
| AY-31637 | about 0.5 to 200 mg/kg body | 2.5 to 100 mg/kg body |
| | weight of the patient per day | weight of the patient per day |
| ciglitazone | about 0.25 to 200 mg/kg | about 0.5 to 50 mg/kg body |
| | body weight of the patient | weight of the patient per day |
| | per day | |
| darglitazone | about 0.05 to 50 mg/kg body | about 0.05 to 5 mg/kg body |
| | weight of the patient per day | weight of the patient per day |
| DN-108 | about 0.25 to 200 mg/kg | about 5 to 100 mg/kg body |
| | body weight of the patient | weight of the patient per day |
| | per day | |
| englitazone | about 0.05 to 50 mg/kg body | about 0.05 to 5 mg/kg body |
| | weight | weight |
| glibenclamide | about 0.1 to 25 mg/day | about 1.75 to 10.5 mg/day |
| glibomuride | about 5 to 150 mg/day | about 12.5 to 75 mg/day |
| gliclazide | about 20 to 480 mg/day | about 80 to 240 mg/day |
| glimepiride | about 0.25 to 12 mg/day | about 1 to 6 mg/day |
| gliquidone | about 5 to 250 mg/day | about 30 to 120 mg/day |
| glisoxepid | about 0.5 to 25 mg/day | about 2 to 16 mg/day |
| incretin hormone like GLP-1 | about 20 to about 100 μg per | |
| | day | |
| KRP297 | about 0.1 to 2500 mg/day | about 1 to 1000 mg/day |
| MCC555 | about 0.1 to 2000 mg/day | about 0.5 to 100 mg/day |
| metformin | about 250 to 1500 mg/day | about 500 to 1250, e.g. |
| | | 1000, mg/day |
| miglitol | about 50 to 500 mg/day | about 100 to 300 mg/day |
| pioglitazone | about 0.1 to 1000 mg/day | about 10 to 150, for example |
| | | 15, 30, 45 or 90, mg/day |
| rosiglitazone | about 0.1 to 500 mg/day | about 1 to 20, for example 1, |
| | | 2, 4 or 8, mg/day |
| T-174 | about 0.1 to 2500 mg/day | about 1 to 1000 mg/day |
| tolbutamide | about 250 to 3000 mg/day | about 1000 to 2000 mg/day |
| troglitazone | about 0.1 to 2000 mg/day | about 50 to 1000 for example |
| | | 100, 200, 400, 600 or 800, |
| | | mg/day, mg/day |
| 5-[3-(4-chlorophenyl])-2- | about 0.1 to 2500 mg/day | about 1 to 1000 mg/day |
| propynyl]-5-phenylsulfonyl)- | | |
| thiazolidine-2,4-dione | | |
| 5-[3-(4-chlorophenyl])-2- | about 0.1 to 2500 mg/day | about 1 to 1000 mg/day |
| propynyl]-5-(4-fluoro- | | |
| phenylsulfonyl)thiazolidine- | | |
| 2,4-dione | | |

The following Examples illustrates the invention described above; they are not, however, intended to limit the scope of the invention in any way.

### EXAMPLE 1

### Tablets of Nateglinide

108,000 tablets, each which contain 120 mg of nateglinide are prepared as follows:

Composition:

| | | |
|---|---|---|
| nateglinide | 12.960 | kg |
| lactose, NF | 30.564 | kg |
| microcrystalline cellulose, NF | 15.336 | kg |
| povidone, USP | 2.592 | kg |
| croscarmellose sodium, NF | 3.974 | kg |
| colloidal silicon dioxide, NF | 1.382 | kg |
| magnesium stearate, NF | 1.231 | kg |
| coating: opadry yellow | 1.944 | kg |

| | |
|---|---|
| purified water, USP* | Q.S. |

* removed during process

Preparation Process: The microcrystalline cellulose, povidone, part of the croscarmellose sodium, nateglinide and lactose are mixed in a high shear mixer and afterwards granulated using purified water. Alternatively, the microcrystalline cellulose, povidone, a portion of the croscarmellose sodium, nateglinide and lactose are granulated in a collette gral granulator with the addition of purified water. The wet granules are dried in a fluid bed dryer and passed through a screen. The colloidal silicon dioxide and the rest of the croscarmellose sodium are mixed, passed through a screen and blended with the dried granules in a V-blender. The magnesium stearate is passed through a screen, blended with the blend from the V-blender and afterwards the total mixture is compressed to tablets. The opadry yellow is suspended in purified water and the tablets are coated with the coating suspension.

### EXAMPLE 2

### Galenic Formulation of Nateglinide No. 2

intra-granular:

| | | |
|---|---|---|
| nateglinide | 120 | mg |
| lactose monohydrate | 283 | mg |
| microcrystalline cellulose | 142 | mg |
| povidone | 24 | mg |
| croscarmellose sodium | 24 | mg |

extra-granular:

| | | |
|---|---|---|
| croscarmellose sodium | 12.8 | mg |
| magnesium stearate | 11.4 | mg |
| opadry yellow | 18.0 | mg |
| colloidal silicon dioxide | 12.8 | mg |

### EXAMPLE 3

### Tablets of Nateglinide

108,000 tablets, each which contain 120 mg of nateglinide are prepared as follows:

Composition:

| | | |
|---|---|---|
| nateglinide | 12.960 | kg |
| lactose, NF | 30.564 | kg |
| microcrystalline cellulose, NF | 15.336 | kg |
| povidone, USP | 2.592 | kg |
| croscarmellose sodium, NF | 3.974 | kg |
| colloidal silicon dioxide, NF | 1.382 | kg |
| magnesium stearate, NF | 1.231 | kg |
| coating: opadry yellow | 1.944 | kg |

| | |
|---|---|
| purified water, USP* | Q.S. |

removed during process

Preparation process: The microcrystalline cellulose, povidone, a portion of the croscarmellose sodium, nateglinide and lactose are granulated in a collette gral granulator with the addition of purified water. The wet granules are dried in a fluid bed dryer and passed through a screen. The colloidal silicon dioxide and the rest of the croscarmellose sodium are mixed, passed through a screen and blended with the dried granules in a V-blender. The magnesium stearate is passed through a screen, blended with the blend from the V-blender and afterwards the total mixture is compressed to tablets. The opadry yellow is suspended in purified water and the tablets are coated with the coating suspension. Variants of this process include adding the colloidal silica and the remaining croscarmellose sodium to the second granulator load after drying, then screening together; and combining as many as 3 granulator/drier loads per batch.

1. Combination which comprises nateglinide (I) 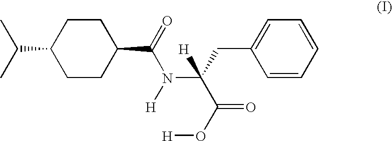
or repaglinide and at least one further antidiabetic compound selected from the group consisting of insulin signalling pathway modulators, compounds influencing a dysregulated hepatic glucose production, pyruvate dehydrogenase kinase (PDHK) inhibitors, inhibitors of gastric emptying, insulin, inhibitors of GSK-3, retinoid X receptor (RXR) agonists, agonists of Beta-3 AR, agonists of uncoupling proteins (UCPs), non-glitazone type PPARγ agonists, dual PPARγ/PPARα agonists, antidiabetic vanadium containing compounds, incretin hormones, β-cell imidazoline receptor antagonists, miglitol and α₂-adrenergic antagonists, in which the active ingredients are present in each case in free form or in the form of a pharmaceutically acceptable salt and optionally at least one pharmaceutically acceptable carrier; for simultaneous, separate or sequential use.

2. Combination according to claim 1 which is a combined preparation or a pharmaceutical composition.

3. Combination according to claim 1 or 2 which is used in the prevention or treatment of diseases.

4. Combination according to any one of claims 1 to 3 wherein the combination comprises nateglinide (I) or a pharmaceutically acceptable salt thereof.

5. Combination according to any one of claim 4, characterized in that nateglinide (I) is present in the B-type or H-type crystal modification.

6. Combination according to any one of claims 1 to 5, characterized in that the combination comprises an incretine hormone.

7. Combination according to any one of claims 1 to 6, characterized in that the combination comprises at least one further pharmaceutically active compound selected from the group consisting of glitazones, sulphonyl urea derivatives, metformin and acarbose, or the pharmaceutically acceptable salts of such compounds.

8. Combination according to any one of claims 1 to 7, characterized in that the combination comprises a compound of formula (II) 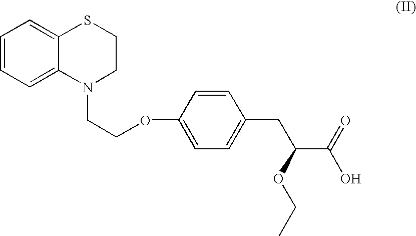
as a dual PPARγ/PPARα agonist.

9. Method of improving the bodily appearance of a mammal which comprises orally administering to said mammal a combination according to any one of claims 1 to 8 in a dosage effective to influence the glucose metabolism, and repeating said dosage until a cosmetically beneficial loss of body weight has occurred.

10. Method of treatment of a warm-blooded animal having metabolic disorders comprising administering to the animal a combination according to any one of claims 1 to 8 in a quantity which is jointly therapeutically effective against metabolic disorders in which the active ingredients can also be present in the form of their pharmaceutically acceptable salts.

11. A pharmaceutical composition comprising a quantity, which is jointly therapeutically effective against metabolic disorders, of a combination according to any one of claims 1 to 8, and at least one pharmaceutically acceptable carrier.

12. Use of a combination according to any one of claims 1 to 8 for the preparation of a medicament for the prevention, delay of progression or treatment of metabolic disorders.

13. Use of a combination according to any one of claims 1 to 8 for the cosmetic treatment of a mammal in order to effect a cosmetically beneficial loss of body weight.

14. A commercial package comprising as active agent a combination according to any one of claims 1 to 8 together with instructions for simultaneous, separate or sequential use thereof in the prevention, delay of progression or treatment of metabolic disorders or in a method of improving the bodily appearance of a mammal.
